(19) United States
(12) Patent Application Publication
(10) Pub. No.: US 2023/0371852 A1
(43) Pub. Date: Nov. 23, 2023

(54) Wide-field system integrating intensity and spatially modulated light for optical tomography and spectroscopy applications

(71) Applicant: Northeastern University, Boston, MA (US)

(72) Inventors: Qianqian Fang, North Reading, MA (US); Miguel Adrian Mireles Nunez, Boston, MA (US); Edward Xu, Boston, MA (US)

(21) Appl. No.: 18/322,418

(22) Filed: May 23, 2023

Related U.S. Application Data
(60) Provisional application No. 63/365,148, filed on May 23, 2022.
(60) Provisional application No. 63/366,615, filed on Jun. 17, 2022.

(51) Int. Cl. A61B 5/1455; G01N 21/49; G01N 21/31; G01N 33/483

(52) U.S. Cl. CPC A61B 5/1455; G01N 21/49; G01N 21/31; G01N 33/4833; G01N 2201/061; A61B 2562/0238

(57) ABSTRACT

Methods and systems for performing optical imaging are provided. An optical imaging system includes a light source configured to generate a spatially-distributed illumination beam of temporally-varying light and a detector configured to detect light resulting from an interaction between the illumination beam and a sample. The system further includes a processor configured to determine spatial and temporal characteristics of the detected light and generate a representation of the sample based on the determined spatial and temporal characteristics.

### RELATED APPLICATIONS

This application claims the benefit of U.S. Provisional Application No. 63/365,148, filed on May 23, 2022 and U.S. Provisional Application No. 63/366,615, filed on Jun. 17, 2022. The entire teachings of the above applications are incorporated herein by reference.

### GOVERNMENT SUPPORT

This invention was made with government support under R01-CA204443 from the National Institutes of Health. The government has certain rights in the invention.

### BACKGROUND

Recent advances in near-infrared (NIR) imaging techniques have generated significant interest for clinical applications, such as cancer screening and neuroimaging, due to the ability to non-invasively assess tissue functional physiology. In many studies, frequency-domain (FD) imaging and spectroscopy using intensity frequency-modulated illumination and phase-resolved detection have been shown to simultaneously recover absolute absorption and scattering tissue properties, which conventional continuous wave (CW) based systems failed to provide.

Recent explorations into enhancing reconstructed optical image resolution and spatial sampling rates have led to the development of high-density diffuse optical tomography (DOT) systems. In recent years, the use of wide-field projection/camera-based or single-pixel imaging systems has achieved significantly enhanced spatial sampling density and large field-of-view. However, a majority of these systems utilize only static CW illumination to perform measurements.

There exists a need for further improved imaging systems that can provide for high-density spatial sampling.

### SUMMARY

Methods and systems are provided that leverage spatially-encoded and temporally-encoded light illumination, via modulations with series of known waveforms and subsequent demodulations of detected light signals with respect to these waveforms, to provide characterizations of optical properties of the target media (e.g., complex biological tissue samples). The simultaneous use of spatial and temporal encoding schemes enables rapid optical scanning of large field-of-view of the sample with high spatial sampling density as well as a rich set of optical properties that conventional continuous wave (CW) based imaging systems fail to capture.

In example systems and methods, high-density frequency-domain (FD) imaging instrumentation and projector-based wide-field illumination techniques are provided that can combine temporal radio-frequency (RF) intensity modulation with spatial-modulation within a single compact system. Conventional FD imaging systems are currently limited to fiber-coupled light delivery and detection and are highly time-consuming to scan a large tissue surface with high spatial density. The provided wide-field frequency-domain (wfFD) systems can achieve simultaneous high-density spatial sampling, which can yield significantly higher signal-to-noise ratios (SNR) and scanning speed compared to traditional fiber-based or point-scanning FD systems, and simultaneous amplitude/phase measurements to enable image reconstructions of both absorption and scattering contrasts.

An optical imaging system includes a light source configured to generate a spatially-distributed illumination beam of temporally-varying light and a detector configured to detect light resulting from an interaction between the illumination beam and a sample. The system further includes a processor configured to determine spatial and temporal characteristics of the detected light and generate a representation of the sample based on the determined spatial and temporal characteristics.

The light resulting from the interaction can be light transmitted through the sample. Alternatively, or in addition, the light can be light reflected by the sample. This approach can be used to characterize both low-scattering and high-scattering. The detected light can be diffuse light. The spatially-distributed illumination beam can comprise a wide-field illumination beam (e.g., can be delivered in a non-contact fashion). A detector, such as a digital micromirror device (DMD) or a camera, can obtain measurements without needing to be in contact with the sample.

Spatial and temporal variations of the illumination beam can be according to a modulation function (including a set of modulation functions). For example, the temporally varying light can comprise intensity-modulated light, and the temporal characteristics can include frequency domain (FD) data for the sample. The processor can be further configured to demodulate the detected light to determine the characteristics based on the modulation function. Arbitrary temporal basis functions can be used to modulate and demodulate the optical signals. In an example, the system includes radio-frequency (RF) modulated light source(s) for FD measurements to provide both amplitude and phase measurements, from which tissue absorption and scattering can be separated. The processor can be further configured to spatially resolve the obtained frequency domain data to generate the representation of the sample. The representation can include, for example, a tomographic reconstruction of the sample.

The light source can include a projector configured to project light according to a source pattern to generate a series of spatially-modulated illumination beams. Optionally, the detector can be a collector configured to collect light from the sample using a detection pattern that is controlled independently of the source pattern. The detection pattern can be complementary to the source pattern (e.g., identical to the source pattern, a mirror image of the source pattern, or having a varying geometry).

The temporally-varying light can include intensity-modulated and spatially-modulated light. Optionally, the projector light source can be configured to project light of at least two fixed wavelengths. An intensity of the projected light of each wavelength can be modulated at a distinct modulation frequency. The wavelength(s) of the projected light can be wavelengths associated with detection of a dynamic physiological property of tissue, such as, for example, a concentration of deoxygenated hemoglobin (Hb), oxygenated hemoglobin (HbO2), water, and lipids.

The detector can be a wide-field sensor, such as a single-pixel sensor.

The representation of the sample can be or include an absorption map, a scattering map, or a combination thereof.

A method of imaging a sample includes generating a spatially-distributed illimitation beam of temporally-modulated light, detecting light resulting from an interaction between the illumination beam and a sample, and determining spatial and temporal characteristics of the detected light. The method further includes generating a representation of the sample based on the determined spatial and temporal characteristics.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of an example configuration for an optical imaging system.

FIG. 2 is a schematic of another example configuration for an optical imaging system.

FIG. 3A is a plot illustrating a conventional, point source FD imaging domain configuration with respect to a sample.

FIG. 3B is a plot illustrating a wide-field FD imaging domain configuration with respect to a sample.

FIG. 3C illustrates a simulated imaging domain with absorbing and scattering inclusions.

FIG. 3D is a cross section of the reconstructed absorption volume of the simulated imaging domain of FIG. 3C using a wide-field FD imaging domain as shown in FIG. 3B.

FIG. 3E is a cross section of the reconstructed scattering volume of the simulated imaging domain of FIG. 3C using a wide-field FD imaging domain as shown in FIG. 3B.

FIG. 4A is a plot illustrating an experimental imaging domain with a wide-field source.

FIG. 4B is a schematic of projected source patterns for an experiment involving the imaging domain of FIG. 4A.

FIG. 4C is an illustrative example plot of the detected amplitude for one of the detectors in the experimental setup of FIGS. 4A and 4B.

FIG. 4D is an illustrative example plot of the detected phase for one of the detectors in the experimental setup of FIGS. 4A and 4B.

FIG. 4E is a cross section of the reconstructed absorption volume of the experimental imaging domain of FIG. 4A.

FIG. 4F is a cross section of the reconstructed scattering volume of the experimental imaging domain of FIG. 4A.

FIG. 4G is a plot of absorption and scattering profiles from the experiment of FIGS. 4A and 4B.

FIG. 5A is an example source pattern, specifically, a single bar pattern, as provided for an experiment.

FIG. 5B is a plot of the spatially-resolved measured amplitude when projecting the source pattern of FIG. 5A onto an optical phantom.

FIG. 5C is a plot of the spatially-resolved simulated amplitude when projecting the source pattern of FIG. 5A onto a simulated optical phantom with the same optical properties of that measured in FIG. 5B.

FIG. 5D is a plot of the spatially-resolved measured phase when projecting the source pattern of FIG. 5A onto an optical phantom.

FIG. 5E is a plot of the spatially-resolved simulated phase when projecting the source pattern of FIG. 5A onto a simulated optical phantom with the same optical properties of that measured in FIG. 5D.

FIG. 6A is a simulated imaging domain using 32×32 point-source (arrows)/detector arrays.

FIG. 6B is a simulated imaging domain using a 32×32 wide-field optimized pattern-based source (square)/detector.

FIG. 7 is a flow diagram illustrating an image processing pipeline for an example system.

### DETAILED DESCRIPTION

A description of example embodiments follows.

Methods and systems leveraging wide-field diffuse optical tomography (wfDOT) with a frequency domain (FD) system to produce a combined spatially- and intensity-modulated wide-field frequency domain (wfFD) tomography system are provided. The systems are aimed to achieve wide-field ultra-high-density spatial sampling of amplitude and phase measurements to enable three-dimensional localization of the tissue optical properties within a single measurement set. Moreover, an example embodiment of the wfFD tomography system offers improved flexibility toward its expansion to allocate other diffuse optical imaging techniques, hence permitting the quantification of other relevant physiological parameters.

Further variations and applications of the wfFD system are also provided. For example, the wfFD system can be adapted and/or used to provide for non-diffusive measurements or media, direct contact measurements, or a combination thereof. Tomographic reconstructions of a sample are not required; for example, the system can be used for spectroscopy measurements. The light applied by the system can be in the form of complex waveforms or non-sinusoidal modulations (e.g., digital signal modulation, chirp, and/or other temporal basis functions). The light detected by the system can be by reflection, transmission, or a combination thereof. While frequency modulation is generally described in the provided example embodiments, it should be understood that other high-order signal characteristics can be used instead of or in addition to frequency modulation. For example, burst or single pulses of light can be used as temporal modulation of the light source, whereas time-resolved sensors can be adapted to the wife-field detector, with detection patterns applied (e.g., arbitrary patterns) to reconstruct the sample optical properties.

Diffuse optical imaging technologies provide a unique capability to non-invasively monitor deep tissue physiology by utilizing non-ionizing near-infrared (NIR) light. Diffuse optical spectroscopy (DOS) techniques are commonly employed to derive localized tissue optical properties, absorption (μₐ) and reduced scattering (μₛ′) coefficients, which are directly related to chromophore concentrations (e.g., Hb, HbO₂, H₂O) within the tissue, thereby allowing for quantification of tissue hemodynamics, such as blood volume, oxygen saturation, and blood flow.

Diffuse optical tomography (DOT) is an optical imaging technique that expands the computation of the localized tissue optical properties to a volumetric distribution, thereby providing three-dimensional maps of a distribution of tissue physiology parameters.

Among DOS techniques, frequency domain (FD) spectroscopy is one of the most used methods to compute tissue optical properties by quantifying amplitude and phase shifts of intensity-modulated (˜MHz) near-infrared light due to intrinsic optical properties of the domain. FD instrumentation often relies on usage of optical fibers to deliver and collect light across different locations on the tissue to approximate bulk optical properties from the whole sampled tissue. More advanced FD devices point towards the approximation of tomographic (FD-DOT) systems by allowing the accommodation of high-density arrays (˜hundreds) of source/detector fibers. Such implementations expand the capabilities of FD techniques by granting improved spatially resolved distributions of optical properties, thereby providing for an ability to resolve heterogeneities embedded within the sampled tissue.

However, such FD-DOT instruments require expensive components and advanced instrumentation technology, while also imposing limited sampling rates due to the need to cycle through all available source/detector fiber combinations. Moreover, the ability of such systems to accurately resolve the spatial localization of heterogeneities (e.g., tumors or cysts) within the retrieved physiological maps can be highly compromised by an inability to physically accommodate optical fibers.

Along these lines, typical DOT systems have historically shown similar limitations as FD-DOT devices and, not until recent years, significant advances have been made towards the implementation of wide-field illumination and detection schemes to surpass those restrictions through novel wide-field diffuse optical tomography (wfDOT) systems. The wfDOT architecture relies on usage of a projection-based illumination coupled with a projector- or camera-based detection, thereby allowing for concurrent exploitation of the benefits of spatially modulated light sources and compression mechanisms (e.g., single-pixel) for light detection to enhance signal quality while achieving ultra-high-density data acquisition at higher sampling rates. However, wfDOT systems remain incapable of providing optical properties decoupling.

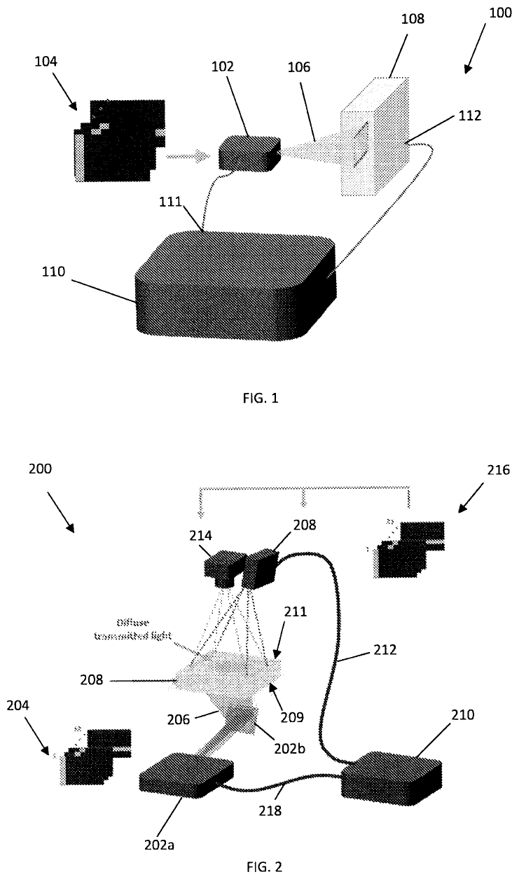

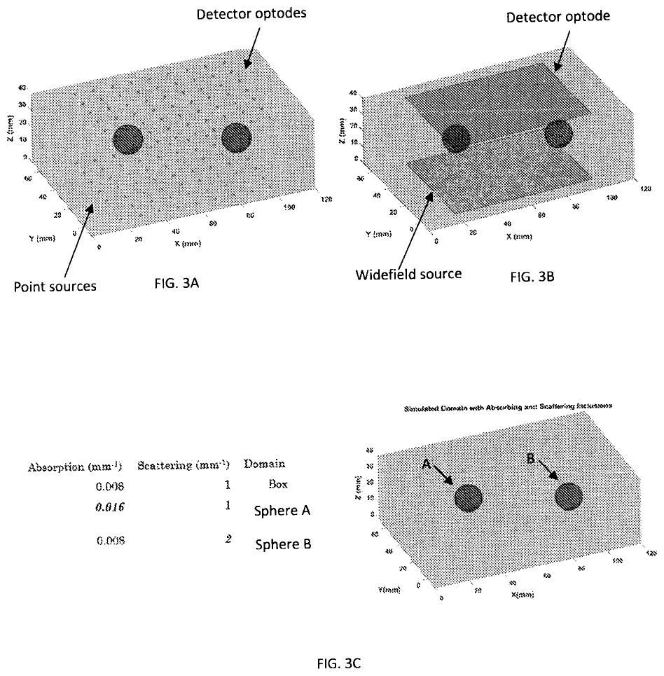

An example optical imaging system is shown in FIG. 1. The system 100 includes a light source 111 to generate temporally-varying illumination (e.g., intensity-modulated light) and a projector 102 that is configured to generate a spatially-distributed illumination beam 106 of light. The system 100 further includes a detector 112 that detects light resulting from an interaction between the illumination beam 106 and a sample 108. As illustrated in FIG. 2, the sample 108 is an optical phantom; however, the sample 108 can be a biological sample (e.g., tissue) or any diffusive media. As illustrated in FIG. 1, the detector 112 is configured to detect light that is transmitted through the sample; however, a detector can be configured to detect light that is reflected from the sample in addition to or in instead of transmitted light. The illumination beam 106 comprises temporally-varying light. In particular, the light provided by the illumination beam can be intensity-modulated, spatially-modulated, or a combination thereof. In lieu of or in addition to intensity-modulated light for frequency domain (FD) data acquisition, other higher order signal characteristics of light can be modulated (e.g., multiplexing of illumination wavelengths by modulation in intensity at different frequencies).

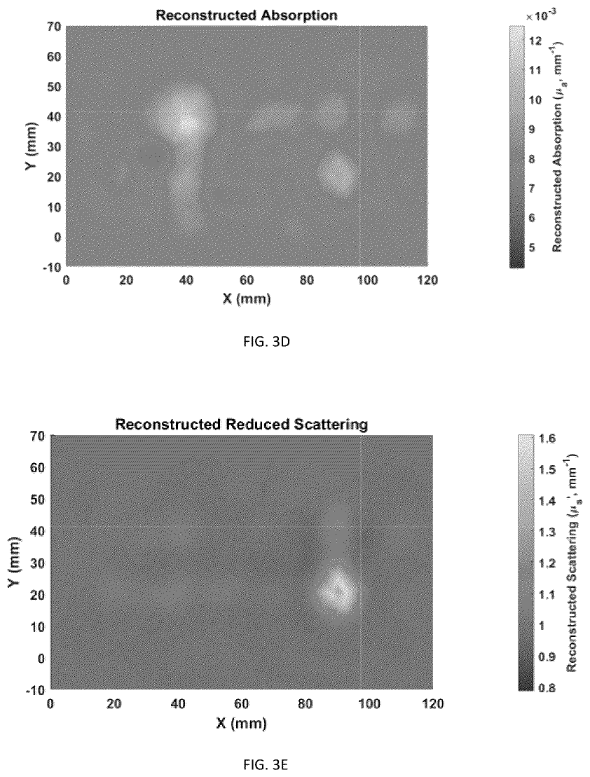

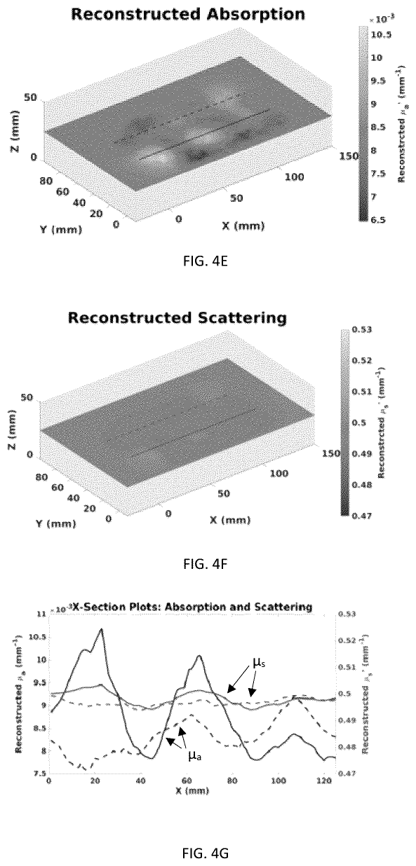

A processor 110 is configured to determine spatial and temporal characteristics of the detected light and generate a representation of the sample based on the determined spatial and temporal characteristics. For example, where the temporally-varying light comprises intensity-modulated light, the processor can be configured to demodulate the detected light to determine the temporal characteristics. Where the temporal characteristics comprise frequency domain (FD) data for the sample, the processor can be configured to spatially resolve the obtained frequency domain data with a tomographic reconstruction of the sample to generate the representation of the sample (see, e.g., FIGS. 3D, 3E and 4E, 4F). The representation of the sample can be an absorption map, a scattering map, or a combination thereof (see, e.g., FIGS. 3D, 3E). Such reconstructed maps can be two-dimensional or three-dimensional.

The light can be delivered by a projector 102 configured to project light according to one or more source patterns 104 to generate a series of illumination beams 106 comprising spatially-modulated light (see, e.g., the projected source patterns of FIG. 4B). The detector 112 can be a wide field detector, such as a single-pixel detector, and/or a single or a bundle of fiber detectors. As illustrated in FIG. 1, the detector 112 is single fiber detector.

Another example of an optical imaging system is shown in FIG. 2. The system 200 includes a light source 202a, 202b. As illustrated, the light source is a digital mirror device (DMD)-based projector 202a and a mirror 202b configured to project an illumination beam 206 onto a sample 208. The light source can be configured to project a series of illumination beams 206 according to a series of source patterns 204 (alternatively referred to as illumination patterns) that provide for spatial modulation of the light with respect to the sample.

One or more laser diodes can be housed within processing device 210 and coupled to projector 202a by way of a source fiber 218 and/or optical coupling (e.g., lens(es)). Together, the processing device 210 and projector 202a are configured to project patterned light at particular wavelengths (e.g., 685 nm, 830 nm, and/or 1,064 nm) onto the sample. Alternatively, the laser diodes can be disposed in a distinct housing or within a same housing as the projector 202a. Light can be projected at one or more wavelengths that is associated with detection of a dynamic physiological property of tissue. For example, a provided wavelength can be one that enables detection of a concentration of deoxygenated hemoglobin (Hb), oxygenated hemoglobin (HbO₂), and/or water. The provided wavelengths can be in the near-infrared range. While laser diodes are described in the provided examples, it should be understood that other originating light sources can be included in lieu of or in addition to laser diodes.

Each of the laser diodes can be modulated by a frequency-domain driver circuit and modulated at a unique RF frequency (e.g., centered around 70 MHz), which can provide for simultaneous multi-wavelength illumination. For example, a frequency-division approach can be used to generate the modulation signals. The laser projector 202a can cast illumination patterns combining all frequencies at a surface 209 of the sample over a given area.

The system 200 further includes a detector 208, which can be a DMD-based detector. Optionally, a second detector 214 is included, which can be a camera, for collection of at least one continuous wave (CW) image per illumination pattern. As illustrated in FIG. 2, the detectors 208, 214 are disposed to detect diffuse light emanating from an opposing surface 211 of the sample. The diffuse-transmitted light can thus be collected in two paths to achieve simultaneous CW and FD data acquisition. The detectors 208, 214 can be configured to collect light according to one or more detection patterns 216. For example, detector 208 can be a DMD-detector with light-coupling optics that is configured to collect RF-modulated light to acquire single-pixel measurements by “projecting” a complementary set of patterns during detection.

With such a configuration, a dot product between the diffuse transmitted light with the pattern basis functions can be performed. Optionally, optical couplers can be used to focus the light signal from the detector-DMD 208 to an optical fiber 212 and guide the light to a light detector sensor, as can be included within processing device 210 (e.g., a modified FD-NIRS system). The frequency-encoded RF signals can be demodulated for each wavelength in real-time using, for example, a field-programmable gate array (FPGA), to obtain both amplitude and phase signals at each wavelength.

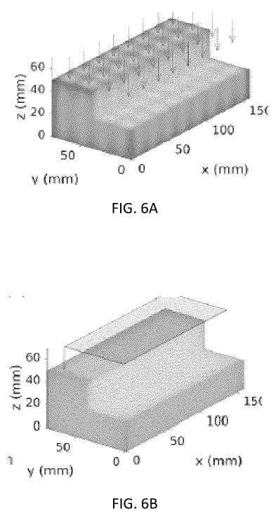

A set of Nₚ illumination patterns (e.g., Nₚ=24 or 32) can be selected, and a same pattern set for detection (N_{d}=Nₚ) can be applied. Using a total of N_{λ} wavelengths, the results can thus yield a total of Nₚ×Nₚ×N_{λ} complex-valued single-pixel readings. A benefit of using a wide-field pattern-based source/detector configuration is uniform sensitivity, as shown in a comparison of FIGS. 6A and 6B. Even with the same number of source and detector pairs, pattern-based measurements yield significantly more uniform sensitivity across the domain compared to point source and detector array configurations.

As used herein, the term “spatially distributed” with respect to an illumination beam means a beam of light that is distributed over a given area with respect to a sample. A “spatially distributed” illumination beam can be, for example, a wide-field beam and/or a beam that is provided by a projector (e.g., as opposed to a point source). A “spatially distributed” illumination beam can be capable of illuminating an entire measurement area at which the sample is disposed but may illuminate less than the entire measurement area due to application of a source pattern that reduces the entire measurement area to a section of the available field-of-view. A source pattern can define a spatial distribution of the illumination beam with respect to the sample.

The provided systems and methods can advantageously enable the projection of simultaneous intensity-modulated light sources onto a tissue surface, hence enabling higher sampling rates for estimating bulk tissue optical properties from the completeness of the illuminated domain while enhancing, by several orders of magnitude, both a signal quality and a sampling density.

As shown in FIG. 3A, traditional fiber-based systems are limited by low spatial sampling density (i.e., a discrete number of fibers), optode-specific fiber coupling coefficients, and expensive (high channel number) instrumentation demands.

In contrast, as shown in FIG. 3B, an example wfFD system includes single source and detector channels that are augmented with DMD-based projection and detection. Such wfFD systems provide for significantly simplified calibration and enable wide-field compressive sensing.

Simulated reconstruction results are shown in FIGS. 3D and 3E for the domain shown in FIG. 3C. As illustrated, the absorbing and scattering inclusions (spheres A and B, respectively) are resolved in the reconstructed maps.

In some configurations, the systems provide for intensity-modulated light-based diffuse optical tomography. By utilizing an intensity-modulated wide-field source coupled with a projector- and/or camera-based detection, tomographic reconstructions can be readily available based on the computation of the amplitude and phase of the detected FD light with respect to the source FD light.

Volumetrically co-registered bulk tissue optical properties and tomographic maps can be generated by the provided systems and methods. In particular, a wfFD system allows for the computation of tissue bulk optical properties from a same volumetric sample as the one being reconstructed with a DOT method, thereby being able to account for tissue heterogeneities that may disrupt accurate optical properties estimation.

The provided systems and methods can also provide for the capability to compute speckle contrast values, either in transmission or in reflection geometries, not only on the amplitude domain but also in the phase domain, hence enabling the quantification of dynamic changes (e.g., blood flow) in the measured samples.

Image reconstructions performed with such systems can provide for an enhanced spatial location and contrast of the embedded heterogeneities in the sample.

The provided systems and methods can be used as an imaging tool for a variety of applications, including, for example: research (e.g., an imaging benchmark system for characterizing a sample's optical properties), breast imaging (e.g., breast tomography equipment, clinical equipment for longitudinal monitoring of therapy outcomes, portable systems for monitoring breast tissue lesions, integration with commercial x-ray imagers for providing supplementary breast hemodynamics information), neurology (e.g., stroke recovery monitoring, ischemic stroke monitoring, behavior responses under specific scenarios, epilepsy), brain-computer interfaces (e.g., gaming), and tissue recovery assessments (e.g., diabetic foot monitoring, burn wounds recovery monitoring) among others.

Additional example embodiments of optical imaging systems and results obtained from simulations and prototype systems are described throughout the Exemplification system herein.

### EXEMPLIFICATION

### Example 1. Combined Intensity- and Spatially-modulated Wide-field Diffuse Optical Tomography

The feasibility of a combined intensity- and spatially-modulated wide-field diffuse optical tomography approach was evaluated with numerical simulations using a MATLAB DOT toolbox “Redbird-m”, an in-house finite-element diffusion-based forward/inverse solver that supports both pattern-illumination and radio frequency (RF) light modulation and detection. A slab of thickness 40 mm with two spherical inclusions (radius 7.5 mm, 20 mm depth) of 2× optical contrast in μₐ and 1.5× contrast in μ′ₛ was simulated. Wide-field sources and point detectors were positioned on opposite faces of the phantom. Using an iterative Gauss-Newton reconstruction algorithm, the bulk optical properties of the domain were recovered from random initial guesses and then absorption and scattering maps were generated.

To experimentally validate the concept, a prototype wfFD system was developed (as illustrated in FIG. 1) that used a 550 μm optical fiber to couple light from a dual-wavelength FD-NIR source with the DMD of a projector. Two laser diodes (690 nm and 830 nm) modulated at 67.5 and 75 MHz, respectively were used as the light source and projected to an area of size 12.5 cm×6 cm by the DMD. A laptop was used to control the projector to project user-defined illumination patterns. Light transmitting through the medium was then collected by a 2.5 mm fiber bundle and routed to a phase-sensitive, avalanche photodiode-based detector unit, the resulting signal was then demodulated to obtain amplitude and phase components. A 4×6 rectangular array of detector locations is drilled into an acrylic plate to position the fiber bundle, with 2 cm spacing separating each. Initial in vitro validation of wfFD measurements were carried out using a silicone-based phantom mimicking physiological optical properties (μₐ=0.03 cm⁻¹, μ′ₛ=9.1 cm⁻¹).

The bulk properties and optical property maps recovered from simulated experiments support the feasibility of simultaneous intensity- and spatially-modulated tomography. Though the simulations were carried out in noise-free conditions, recovered bulk properties using randomized initial guesses fell within 1% of the true background absorption and scattering. Reconstructed inclusions were correctly localized, with a full-width half maximum of 17 mm, a 1.13× increase from that of the simulated inclusion.

In vitro phantom measurements provided further evidence that phase and amplitude trends seen in simulations are reproducible under experimental conditions. The spatially varying contour plots for experimental and modeled data, simulated using similar optical properties, provide a representative comparison of amplitude decay and phase shift trends over the array of detector positions. Similar trends were observed with distance from the source pattern, providing confidence for tomographic image reconstruction using experimental data.

### Example 2. Ultra-high-density Frequency-domain System

A frequency-domain (FD) system comprising two multiplexed lasers sources at 690 (HL6750MG, Thorlabs GmbH, Germany) and 830 nm (HL8338MG, Thorlabs GmbH, Germany) modulated at 67.5 MHz and 75 MHz, respectively, was custom-built. The laser diodes were coupled to each of the inputs of a 550 μm core bifurcated optical fiber bundle (BFY400LS02, Thorlabs, GmbH, Germany). The single output fiber was attached to a fix focus optical collimator (FS220SMA-780, Thorlabs, GmbH, Germany) for producing a slightly expanding beam. The output fiber was then coupled with a digital micro-mirror device (DMD) based projector (P300, AAXA Technologies, USA). The triplet LED light sources, the collimation lenses and the dichroic mirrors built-in the projector were removed to allow for a direct optical coupling of the collimated fiber bundle output light. The beam size tuned to fully illuminate the projector's DMD sensor after passing through the internal optical prism. The light being reflected by the DMD array was then collected and expanded by the built-in optical lenses array. This configuration allowed for a wide-field intensity- and spatially-modulated illumination area of 12×6 cm when projecting at about 15 cm distance to the sample's surface. This light source was projected onto the sample's surface and the diffuse transmitted light was collected by a second DMD-based projector by making use of the reciprocity concept in which, same optical path should be followed by the light. The collected diffuse transmitted light by the expansion lenses was projected onto the DMD sensor and then reflected to an optical collimator, coupling it into a secondary detection 400 μm core optical fiber. The detection fiber was attached to a FD processing board using a high-speed optical detector (9C5331-04, Hamamatsu, Japan). Recorded optical signals were then post-processed for retrieving amplitude and phase-shift metrics for each source wavelength.

The architecture allowed for the introduction of, apart from a spatially modulated FD light source that can be further optimized to adapt to the targeted domain (see Mireles M, Xu E, Ragunathan R, and Q Fang, “Target-adaptive, compressive and ultra-high-density wide-field diffuse optical tomography,” in Optica: Biomedical Optics Congress, 2022), a physical single-pixel data compression mechanism in the detection side by means of the secondary DMD-based projector to improve signal-to-noise ratio while providing ultra-higher-density and faster domain sampling.

It is noted that the provided detection approach can also be replaced by a fiber-based detection (as shown in the schematic of FIG. 1) with a potential drawback of reducing a sampling density and the usage of single-pixel data compression, but is still capable of providing a benefit by way of an improved signal-to-noise ratio due to the wide-field illumination and a faster data acquisition respect to solely fiber-based FD systems.

### Example 3. Wide-field Tomography System

A wide-field tomography system using the modified projector described in Example 2 and coupled with an FD light source was used to produce pattern light illumination on a sample's surface. However, unlike projector-based detection required for FD data collection, the wide-field DOT system utilizes an EMCCD camera (Andor Luca R, Oxford Instruments, UK) to acquire images of the diffuse transmitted light produced by each source pattern. The collection of images was processed offline where, in contrast with DMD-based data acquisition, a digital detection pattern was applied for retrieving single-pixel values.

Like the above-described FD system, the wide-field pattern illumination approach allowed for ultra-high-density and faster domain sampling.

### Example 4. An Integrated Wide-field Frequency Domain (wfFD) System

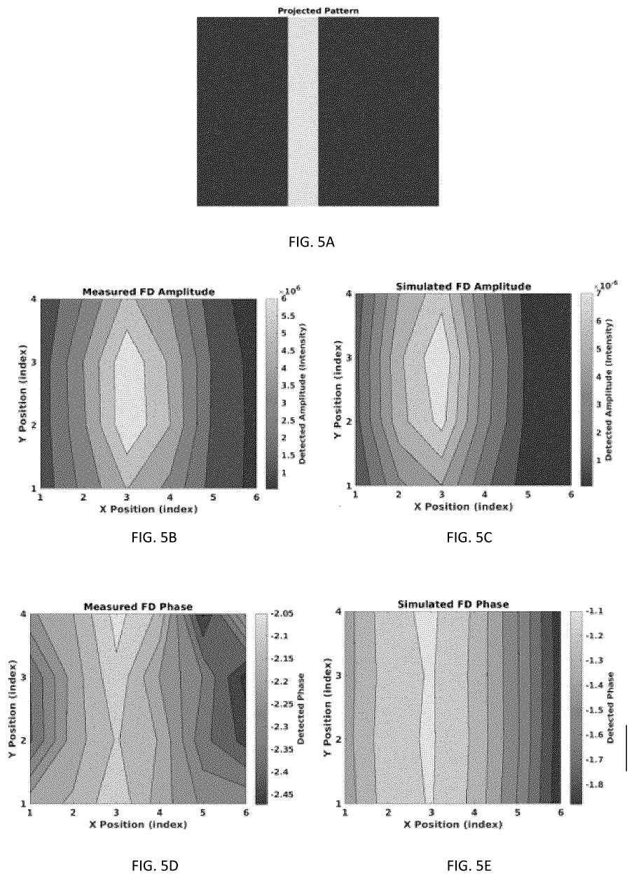

The above-described systems, Ultra-High-Density FD (Example 2) and wide-field tomography (Example 3) were then hybridized into a single, bed side and compact device with a unified hardware controlling and data acquisition software. The system provides the capability to spatially resolved light amplitude and phase shifts, as shown in FIGS. 5A-5E.

The simulated and measured spatially resolved amplitude and phase from a single bar wide-field source illumination pattern are shown in FIGS. 5A-5E. The expected spatial distribution of the light amplitude (FIG. 5C) and phase (FIG. 5E) from a single bar pattern source (FIG. 5A) are contrasted with a fiber-based measurement of the amplitude (FIG. 5B) and phase (FIG. 5D) of a real optical phantom with same optical properties as the simulated domain.

The introduced system allows for the implementation of other diffuse optical techniques, such as speckle contrast optical spectroscopy (SCOS) and speckle contrast optical tomography (SCOT), with minimal to null hardware modifications. In addition, by providing supplementary information as the spatially resolved phase shift, further constraints on the inversion problem can be performed, providing for more accurate three-dimensional physiology maps reconstruction.

### Example 5. Algorithm for Tomographic Data Analysis

Two offline, DOT-based reconstruction workflows were implemented to provide detailed spatial tissue physiology maps. In this regard, the focus was mainly on breast optical imaging applications given the recently developed optical mammography co-imager (OMCI) device, which is readily scalable to apply the proposed wfFD technology. Therefore, for subjects with a previously acquired structural x-ray mammography scans, subject-specific anatomical models were developed and run an iterative Gauss-Newton reconstruction to solve the DOT inverse problem and recover volumetric breast tissue hemodynamics. To this end, the x-ray scans were segmented using SPM which was further represented by a tetrahedral mesh generated using Iso2Mesh platform. Afterwards, CPU-based diffuse equation simulations with the photon-replay algorithm were performed using the newly developed Redbird-m toolbox to create the Jacobians corresponding to the source pattern and the corresponding fiber detector for FD and pattern detector for DOT, respectively. A multi-linear singular value decomposition (MLSVD) method was applied to remove spatial noise from the collected DOT images. On the other hand, for subject with no available x-ray scans, a generic breast mesh template was used to minimize reconstruction errors due to boundary effects.

To further improve the contrast and resolution of tissue structures in the reconstructed maps, the recently developed prior-guided template reconstruction algorithm can be utilized. This involves the assumption of Gaussian-spherical inclusions within the breast tissue (e.g., tumors) as probability maps of the tumor location. The spherical template is then scanned across the region of interest in small steps. Enhanced tissue physiology contrast is expected when the spherical matches the location of true tissue heterogeneities. If a single-interaction (i.e., linear) reconstruction is performed, no additional simulations are needed because the Jacobians used in these reconstructions are the same as those in non-prior-guided reconstructions. The regularization matrix L and the solution of the normal equation can be re-evaluated between different speculated tumor locations.

Moreover, the recently developed pattern optimization algorithm based on MLSVD decomposition can also be applied to enhance further the contrast and spatial location of the tissue heterogeneities while minimizing the presence of reconstruction artifacts. It is noted that the same algorithm provides expanded capabilities by, apart from allowing to remove spatial noise and find a target-specific optimized pattern basis function, allowing for the application of compressive sensing mechanisms. This technique provides a robust mechanism for removing low rank information content, reducing the inverse problem size and the computational cost and time to produce a reconstruction while maximizing the information content.

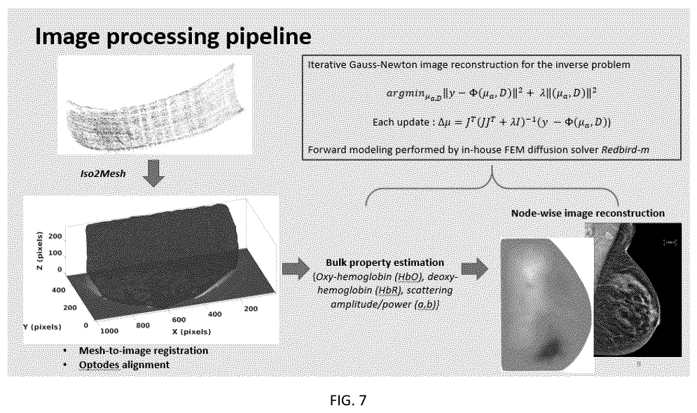

An overview of the image processing pipeline, as applied to breast imaging is shown in FIG. 7.

### Example 6. Heterogeneous Phantom Preliminary Results Using a Prototype Fiber-based wfFD System

Preliminary results from a heterogeneous phantom measurement are shown in FIGS. 4A-4D. In FIGS. 4A and 4B, the measured phantom and the wide-field FD sources patterns are shown. A schematic of the heterogeneous phantom domain consisting of two rows of inclusion sorted by size in opposite directions and the wide-field source and fiber detectors are shown in FIG. 4A. The set of projected binary source patterns are shown in FIG. 4B.

The breast-like heterogeneous phantom consisted of two rows with three inclusions each ordered in size in opposite directions respect to each other. The inclusions in the front and back row are of 2× and 4× contrast with respect to the background, respectively. The wide-field source area and the fiber detector grid of 4×6 locations with 2 cm inter-spacing are illustrated.

Representative plots of the collected raw amplitude and phase data are shown in FIGS. 4C and 4D for all single bar pattern sources and for a single fiber-based detector. The amplitude (FIG. 4C) and phase (FIG. 4D) data collected by a single fiber detector at a given position from each of the wide-field FD source patterns are shown. The shadowed area represents the distance-based thresholding data to be used for tomographic reconstructions.

The reconstructed three-dimensional maps from the heterogeneous phantom are shown in FIGS. 4E-4G. The feasibility of recovering most of the expected inclusions within the heterogeneous domain with acceptable contrast levels and localization, as depicted in the cross-section plot, is shown. The reconstructed absorption map is shown in FIG. 4E. The reconstructed reduced scattering map in shown in FIG. 4F. A cross-section plot at each row of the heterogeneous inclusions is shown in FIG. 4G.

The results indicate a successful reconstruction of all but the smallest inclusion (1 cm) with lower contrast from background (2×). Minimal changes in scattering, in agreement with presence of absorbing inclusions, were observed. Relative contrast and size of recovered inclusions corresponds with expectations.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. An optical imaging system, comprising:
a light source configured to generate a spatially-distributed illumination beam of temporally-varying light;
a detector configured to detect light resulting from an interaction between the illumination beam and a sample; and
a processor configured to:
determine spatial and temporal characteristics of the detected light; and
generate a representation of the sample based on the determined spatial and temporal characteristics.

2. The optical imaging system of claim 1, wherein the light resulting from the interaction is light transmitted through the sample.

3. The optical imaging system of claim 1, wherein the detected light is diffuse light.

4. The optical imaging system of claim 1, wherein the spatially-distributed illumination beam comprises a wide-field illumination beam.

5. The optical imaging system of claim 1, wherein spatial and temporal variations of the illumination beam are according to a modulation function, and wherein the processor is further configured to demodulate the detected light to determine the characteristics with based on the modulation function.

6. The optical imaging system of claim 5, wherein the temporal characteristics comprise frequency domain data from the sample, and wherein the processor is further configured to spatially resolve the obtained frequency domain data to generate the representation of the sample.

7. The optical imaging system of claim 1, wherein the light source comprises a projector configured to project light according to a source pattern to generate a series of spatially-modulated illumination beams.

8. The optical imaging system of claim 7, wherein the detector is a collector configured to collect light from the sample using a detection pattern controlled independently of the source pattern.

9. The optical imaging system of claim 1, wherein the temporally-varying light comprises intensity-modulated and spatially-modulated light.

10. The optical imaging system of claim 1, wherein the light source comprises a projector configured to project light of at least two fixed wavelengths.

11. The optical imaging system of claim 10, wherein the light source is configured to modulate an intensity of the projected light of each wavelength at a distinct modulation frequency.

12. The optical imaging system of claim 1, wherein the light source is configured to project light of a wavelength associated with detection of a dynamic physiological property of biological tissue.

13. The optical imaging system of claim 12, wherein the wavelength provides for detection of a concentration of at least one of deoxygenated hemoglobin (Hb), oxygenated hemoglobin (HbO₂), water (H₂O), and lipids.

14. The optical imaging system of claim 1, wherein the detector is a wide-field sensor.

15. The optical imaging system of claim 14, wherein the wide-field sensor is a single-pixel sensor.

16. The optical imaging system of claim 1, wherein the representation of the sample comprises an absorption map, a scattering map, or a combination thereof.

17. A method of imaging a sample, comprising:
generating a spatially-distributed illumination beam of temporally-varying light;
detecting light resulting from an interaction between the illumination beam and a sample;
determining spatial and temporal characteristics of the detected light; and
generating a representation of the sample based on the determined spatial and temporal characteristics.

18. The method of claim 17, wherein detecting light comprises detecting light transmitted through the sample.

19. The method of claim 17, wherein detecting light comprises detecting diffuse light.

20. The method of claim 17, wherein generating the spatially-distributed illumination beam comprises generating a wide-field illumination beam.

21. The method of claim 17, wherein spatial and temporal variations of the illumination beam are according to a modulation function, and wherein the method further comprises demodulating the detected light to determine the characteristics based on the modulation function.

22. The method of claim 21, wherein the temporal characteristics comprise frequency domain data for the sample, and wherein the method further comprises spatially resolving the obtained frequency domain data with a tomographic reconstruction of the sample to generate the representation of the sample.

23. The method of claim 17, wherein generating the spatially-distributed illumination beam comprises projecting light according to a source pattern to generate a series of spatially-modulated illumination beams.

24. The method of claim 23, wherein detecting light comprises collecting light from the sample using a detection pattern controlled independently of the source pattern.

25. The method of claim 17, wherein the temporally-varying light comprises intensity-modulated and spatially-modulated light.

26. The method of claim 17, wherein generating the spatially-distributed illumination beam comprises projecting light of at least two fixed wavelengths.

27. The method of claim 26 further comprising modulating an intensity of the projected light of each wavelength at a distinct modulation frequency.

28. The method of claim 17, wherein generating the spatially-distributed illumination beam comprises projecting light of a wavelength associated with detection of a dynamic physiological property of tissue.

29. The method of claim 28, wherein the wavelength provides for detection of a concentration of at least one of deoxygenated hemoglobin (Hb), oxygenated hemoglobin (HbO₂), water (H₂O), and lipids.

30. The method of claim 17, wherein generating the representation of the sample comprises generating an absorption map, a scattering map, or a combination thereof.
